(19) United States
(12) Patent Application Publication
(10) Pub. No.: US 2011/0015261 A1
(43) Pub. Date: Jan. 20, 2011

(54) USE OF A COMPOSITION COMPRISING AT LEAST ONE BETA-BLOCKER FOR THE TREATMENT OF SLEEP DISORDERS

(75) Inventors: Søren Tullin, Soborg (DK); Birger Jan Olsen, Kobenhavn O (DK)

Correspondence Address: CLARK & ELBING LLP, 101 FEDERAL STREET, BOSTON, MA, 02110, US

(73) Assignee: Zleepax Europe ApS, Kobenhavn (DK)

(21) Appl. No.: 12/864,450

(22) PCT Filed: Jul. 4, 2008
(86) PCT No.: PCT/DK2008/000249

§ 371 Date: Jul. 23, 2010

(30) Foreign Application Priority Data
Aug. 3, 2007 (DK) PA 2007 0116
Sep. 17, 2007 (DK) PA2007 01332
Feb. 11, 2008 (DK) PA 2008 00182
May 2, 2008 (DK) PA 2008 00625
Jul. 4, 2008 (DK) PA 2008 00932

(51) Int. Cl. A61K 31/352; A61K 31/138; A61P 25/00

(52) U.S. Cl. 514/456; 514/652

(57) ABSTRACT

A composition comprising specific beta-blockers such as bisoprolol and nebivolol for the treatment of insomnia and/or another sleep disorder. The composition should be given in such an amount that it causes a less than 40% decrease in the amount of aMT6s in complete nocturnal urin. The composition can be a combination treatment comprising a specific beta-blocker in combination with another known drug e.g., melatonin with similar effect for treatment of insomnia.

### BACKGROUND OF THE INVENTION

### Current Medical Treatments for Insomnia (Adapted from Wikipedia)

Many insomniacs rely on sleeping tablets and other sedatives to get rest. All sedative drugs have the potential of causing psychological dependence where the individual cannot psychologically accept that they can sleep without drugs. Certain classes of sedatives such as benzodiazepines and newer nonbenzodiazepine drugs can also cause physical dependence which manifests in withdrawal symptoms if the drug is not carefully titrated down.

The most commonly used class of hypnotics prescribed for insomnia are the benzodiazepines. These medications can develop tolerance and dependence, especially after consistent usage over long periods of time.

Nonbenzodiazepine prescription drugs, including the nonbenzodiazepines zolpidem and zopiclone appear to cause both psychological dependence and physical dependence, and can also cause the same memory and cognitive disturbances as the benzodiazepines along with morning sedation. Some antidepressants such as mirtazapine, trazodone and doxepin have a sedative effect, and are prescribed off label to treat insomnia. The major drawback of these drugs is that they have antihistaminergic, anticholinergic and antiadrenergic properties which can lead to many side effects. Some also alter sleep architecture.

Melatonin has proved effective for some insomniacs in regulating the sleep/waking cycle, but lacks definitive data regarding efficacy in the treatment of insomnia. Melatonin agonists, including Ramelteon (Rozerem), seem to lack the potential for abuse and dependence. This class of drugs has a relatively mild side effect profile and lower likelihood of causing morning sedation.

The antihistamine diphenhydramine is widely used in nonprescription sleep aids. While it is available over the counter, the effectiveness of these agents may decrease over time and the incidence of next-day sedation is higher than for most of the newer prescription drugs.

Dependence does not seem to be an issue with this class of drugs. Low doses of certain atypical antipsychotics such as quetiapine (Seroquel) are also prescribed for their sedative effect but the danger of neurological and cognitive side effects make these drugs a poor choice to treat insomnia.

Some insomniacs use herbs such as valerian, chamomile, lavender, hops, and passion-flower. Valerian has undergone multiple studies and appears to be modestly effective. Cannabis has also been suggested as a treatment for insomnia.

Though Alcohol may have sedative properties, the REM sleep suppressing effects of the drug prevent restful, quality sleep. Also, middle-of-the-night awakenings due to polyuria or other effects from alcohol consumption are common, and hangovers can also lead to morning grogginess.

### Unmet Medical Need

As can be seen from this short review of the current medical treatments for insomnia, there is a huge unmet medical need for an efficacious treatment of insomnia (and other sleep disturbances) which does not cause psychological/physical dependence, morning sedation, neurological/cognitive side effects and/or many other side effects. The current invention addresses this unmet medical need by providing a novel safe and efficacious treatment for insomnia (and other sleep disturbances), without any of the side effects of the current treatments.

### Beta-Blockers and Sleep Disturbances

Beta-blockers are notorious for causing sleep disturbances and nightmares, presumably because they inhibit the nocturnal Melatonin secretion (“Treatment with beta-adrenoceptor blockers reduces plasma melatonin concentration”. P. J. Cowen et al., Br J Clin Pharmacol, Vol. 19 (2), 258-260, 1985). Analysis of the melatonin metabolite 6-sulfatoxy-melatonin (aMT6s) in urine from healthy volunteers, has e.g. shown that the beta-blockers S-Propranolol (40 mg dose) and S-Atenolol (50 mg dose) cause an impressive 80-90% decrease in the nocturnal aMT6s secretion 12 hours after taking the drug (“Influence of beta-blockers on melatonin release”. K. Stoschitzky et al., Eur J Clin Pharmacol, Vol. 55, 111-115, 1999). Given that Melatonin plays a role in sleep induction and exerts various effects on circadian rhythm, it seems plausible that the sleep disturbances caused by beta-blockers are at least partly caused by their effects on the Melatonin levels. In contrast to the findings with S-Propranolol and S-Atenolol, a recent study has shown that the 3rd generation beta-blockers Carvedilol and Nebivolol have little if any effects on the nocturnal aMT6s urinary secretion in healthy volunteers (“Comparing Beta-Blocking Effects of Bisoprolol, Carvedilol and Nebivolol”. K. Stoschitzky et al., Cardiology, Vol. 106, 199-206, 2006). Moreover, the same study shows that Carvedilol, Nebivolol and Bisoprolol have no negative effect on the quality of sleep in patients with hypertension. In contrast to this finding, a more recent publication suggests that Nebivolol can improve the quality of sleep in patients with hypertension (“Nebivolol is Different From Atenolol in Terms of Impact Onto Sleep”. A. Erdem et al., The Anatolian Journal of Clinical Investigation, Vol. 1(1), 25-29, 2007). The authors conclude that “the improvement of sleep quality in the Nebivolol group might well be due to simply blood pressure control and lack of central side effect of the drug”. As support for this conclusion it is well known that hypertension is associated with poor quality of sleep (Prejbisz et al., Blood Pressure. Vol. 15, 213-219, 2006). Moreover, other anti-hypertensive drugs have also been shown to improve the quality of sleep in patients with hypertension. The ACE inhibitor Captopril has e.g. been shown to improve the quality of sleep in patients with hypertension (“Quality of Life and Antihypertensive Therapy in Men—A Comparison of Captopril with Enalapril”. M. A. Testa et al., The New England Journal of Medicine Vol. 328, 907-913, 1993). The beneficial effect of the anti-hypertensive 3^{rd} generation beta-blocker Nebivolol on sleep quality in patients with hypertension (observed in Erdem's paper but not in other publications) is accordingly most likely caused by the blood pressure reduction induced by the drug.

The importance of norepinephrine in the regulation of sleep, has been studied in norepinephrine-deficient mice. The study suggests that norepinephrine is wake promoting after a mildly stressful event (“Norepinephrine-deficient mice exhibit normal sleep-wake states but have shorter sleep latency after mild stress and low doses of amphetamine”, M. S. Hunsley and R. D. Palmiter, Sleep, Vol. 26 (5), 521-526, 2003). In man it has been shown that poor sleep (in stressed elderly caregivers), is associated with an increased plasma norepinephrine concentration. (“Sleep Disturbance, Norepinephrine, and D-Dimer Are All Related in Elderly Caregivers of People With Alzheimer Disease”, B. T. Mausbach et al, Sleep, Vol. 29(10), 1347-1352, 2006).

### DEFINITIONS

It is believed that the present invention will be better understood from the following definitions.

As used herein aMT6s refers to the melatonin metabolite: 6-sulfatoxy-melatonin.

As used herein Quality of Sleep might be measured by employing the Pittsburgh Sleep Quality

Index (“The Pittsburgh Sleep Quality Index: A New Instrument for Psychiatric Practice and Research”, D. J. Buysse et al., Psychiatry Res Vol. 28, 193-213, 1989)

As used herein “Stress” refers to:

An emotionally disruptive or upsetting condition occurring in response to adverse external influences and capable of affecting physical health which can be characterized by increased heart rate, a rise in blood pressure, muscular tension, irritability, insomnia and depression. Examples of stressful life events include, but are not limited to: Death of spouse, Divorce, Marital separation, Jail term or death of close family member, Personal injury or illness, Loss of job due to termination, Marital reconciliation or retirement, Pregnancy and Change in financial state (negative).

As used herein, “comprising” means that other steps and/or ingredients can be added.

As used herein beta-blocker refers to:

Antagonists (full or partial) of beta-adrenergic receptors. Some beta-blockers antagonize one specific subtype of beta-adrenergic receptors (e.g. a beta 1 selective beta-blocker which selectively antagonizes the beta-1 adrenergic receptor), whereas other beta-blockers are non-selective. In context of this invention the term “beta-blocker” refers to all types of antagonists of beta-adrenergic receptors, regardless of whether the beta-blocker antagonize one, two or more beta-adrenergic receptors and regardless of whether they affect other processes. Examples of beta-blockers include, but are not limited to: Acebutolol, Atenolol, Betaxolol, Bisoprolol, Bucindolol, Carteolol, Carvedilol, Celiprolol, Esmolol, Labetalol, Metoprolol, Nadolol, Nebivolol, Penbutolol, Pindolol, Propranolol, Timolol.

As used herein beta 1 selective beta-blocker refers to:

Beta-blockers where the IC50 for inhibition of the effect of noradrenaline on the beta 1 adrenergic receptor in a functional assay (e.g. cellular cAMP production) is at least 5 times less than for any other adrenergic receptor.

As used herein complete nocturnal urine refers to:

The total amount of urine that is produced during one night from the time an individual goes to bed until the individual wakes up in the morning.

As used herein complete daytime urine refers to:

The total amount of urine that is produced during one day from the time an individual wakes up in the morning until the individual goes to bed.

As used herein patient refers to:

A person suffering from insomnia or another sleep disorder.

As used herein:

The term“elderly” is intended to mean humans from 65 years and above.

The term“adults” is intended to mean humans from 18 to 64 years.

The term“children” is intended to mean humans from 0 to 17 years.

As used herein insomnia refers to:

The perception or complaint of inadequate or poor-quality sleep because of one or more of the following: difficulty falling asleep; waking up frequently during the night with difficulty returning to sleep; waking up too early in the morning; or unrefreshing sleep. Insomnia is not defined by the number of hours of sleep a person gets or how long it takes to fall asleep. Individuals vary normally in their need for, and their satisfaction with, sleep. Insomnia may cause problems during the day, such as tiredness, a lack of energy, difficulty concentrating, and irritability.

Types of Insomnia: Primary insomnia is associated with complaint in initiating, maintaining or non-restoratively sleep, not exclusively occurring due to another mental disorder, physiological effects of a substance or a general medical condition. Secondary insomnia is associated with complaint in initiating, maintaining or non-restoratively sleep, occurring due to another mental disorder, physiological effects of a substance or a general medical condition.

Insomnia can be classified as transient (short term), intermittent (on and off), and chronic (constant). Insomnia lasting from a single night to a few weeks is referred to as transient. If episodes of transient insomnia occur from time to time, the insomnia is said to be intermittent. Insomnia is considered to be chronic if it occurs on most nights and lasts a month or more.

Causes of Insomnia: Certain conditions seem to make individuals more likely to experience insomnia. Examples of these conditions include: advanced age (insomnia occurs more frequently in those over age 60); female gender; and a history of depression. If other conditions (such as stress, anxiety, a medical problem, or the use of certain medications) occur along with the above conditions, insomnia is more likely.

There are many causes of insomnia. Transient and intermittent insomnia generally occur in people who are temporarily experiencing one or more of the following: stress, environmental noise, extreme temperatures, a change in the surrounding environment, sleep/wake schedule problems such as those due to jet lag, or medication side effects.

Chronic insomnia is more complex and often results from a combination of factors, including underlying physical or mental disorders. One of the most common causes of chronic insomnia is depression. Other underlying causes include arthritis, kidney disease, heart failure, asthma, sleep apnea, narcolepsy, restless leg syndrome, Parkinson disease, and hyperthyroidism. However, chronic insomnia may also be due to behavioral factors, including the misuse of caffeine, alcohol, or other substances; disrupted sleep/wake cycles as may occur with shift work or other nighttime activity schedules; and chronic stress.

Certain Behaviors: Behaviors that perpetuate insomnia in some people include: expecting to have difficulty sleeping and worrying about it, ingesting excessive amounts of caffeine, drinking alcohol or smoking cigarettes before bedtime, excessive napping in the afternoon or evening, and irregular or continually disrupted sleep/wake schedules. These behaviors may prolong existing insomnia, and they can also be responsible for causing the sleeping problem in the first place. Stopping these behaviors may eliminate the insomnia altogether.

As used herein sleep disorder include:
Bruxism: The sufferer involuntarily grinds or clenches his or her teeth while sleeping.
Delayed sleep phase syndrome (DSPS): A sleep disorder of circadian rhythm, characterized by the inability to wake up and fall asleep at the desired times, but not by inability to stay asleep.
Hypopnea syndrome: Abnormally shallow breathing or slow respiratory rate while sleeping.
Narcolepsy: The condition of falling asleep spontaneously and unwillingly at inappropriate times.
Night terror or Pavor nocturnus or sleep terror disorder: abrupt awakening from sleep with behavior consistent with terror.
Parasomnias: Include a variety of disruptive sleep-related events.
Periodic limb movement disorder (PLMD): Sudden involuntary movement of arms and/or legs during sleep, for example kicking the legs. Also known as nocturnal myoclonus. See also Hypnic jerk, which is not a disorder. PLMD sufferers often do not also have RLS.
Rapid eye movement behavior disorder (RBD): Acting out violent or dramatic dreams while in REM sleep.
Hatzfeldt Syndrome or Systemic Neuro-Epiphysial Disorder (SNED) is a somnipathy mainly characterized by an irregular sleep pattern, as well as irregular behavior
Restless legs syndrome (RLS): An irresistible urge to move legs. RLS sufferers often also have PLMD.
Shift work sleep disorder (SWSD).
Sleep apnea: The obstruction of the airway during sleep, causing loud snoring and sudden awakenings when breathing stops.
Sleepwalking or somnambulism: Engaging in activities that are normally associated with wakefulness (such as eating or dressing), which may include walking, without the conscious knowledge of the subject.
Snoring: Loud breathing patterns while sleeping; sometimes this is a symptom of sleep apnea.
Dysomnias—A broad category of sleep disorders characterized by either hypersomnolence or insomnia. The three major subcategories include intrinsic (i.e., arising from within the body), extrinsic (secondary to environmental conditions or various pathologic conditions), and disturbances of circadian rhythm. MeSH
   Insomnia
   Narcolepsy
   Obstructive sleep apnea
   Restless leg syndrome
   Periodic limb movement disorder
   Hypersomnia
      Recurrent hypersomnia—including Kleine-Levin syndrome
      Posttraumatic hypersomnia
      “Healthy” hypersomnia
   Circadian rhythm sleep disorders
      Delayed sleep phase syndrome
      Advanced sleep phase syndrome
   Non-24-hour sleep-wake syndrome

As used herein Bruxism refers to:

Grinding of the teeth, typically accompanied by clenching of the jaw. In most people, bruxism is mild enough not to be a health problem; however, some people suffer from significant bruxism that can become symptomatic. Bruxism often occurs during sleep and can even occur during short naps. Bruxism is one of the most common sleep disorders: 30 to 40 million Americans grind their teeth during sleep.

As used herein hypertension refers to:

“High blood pressure”, HTN or HPN, a medical condition in which the blood pressure is chronically elevated. It was previously referred to as arterial hypertension, but in current usage, the word “hypertension” without a qualifier normally refers to arterial hypertension.

Hypertension can be classified as either essential (primary) or secondary. Essential hypertension indicates that no specific medical cause can be found to explain a patient's condition. Secondary hypertension indicates that the high blood pressure is a result of (i.e. secondary to) another condition, such as kidney disease or certain tumors (especially of the adrenal gland).

As used herein hypertension is considered to be present when the seated systolic blood pressure >140 mmHg and the seated diastolic blood pressure >90 mmHg. As used herein an individual is considered non-hypertensive when the seated systolic blood pressure <140 mmHg or the seated diastolic blood pressure <90 mmHg.

In another embodiment hypertension is considered to be present when the seated systolic blood pressure >140 mmHg and/or the seated diastolic blood pressure >80 mmHg. According to this embodiment an individual is considered non-hypertensive when the seated systolic blood pressure <140 mmHg and the seated diastolic blood pressure <80 mmHg.

### EXAMPLES

### Example 1

6 non-hypertensive stressed individuals with transient insomnia (5 males and 1 female) were treated with 1.25 mg Hypoloc (Nebivolol) 2 hours before bedtime. All individuals reported a significant improvement in sleep quality.

### Example 2

1.25 mg Bisoprolol is given 2 hours before bedtime to stressed individuals suffering from insomnia.

### Example 3

A composition comprising 1.25 mg Hypoloc (Nebivolol) and 1 mg Melatonin is given 1 hour before bedtime to individuals above 55 years of age, who are suffering from insomnia.

### Example 4

10 mg Propal (Propranolol) is given in the morning to individuals suffering from insomnia that is associated with stress.

1. A method of treating insomnia and/or another sleep disorder in an individual or a patient, said method comprising administering to said individual or said patient a composition comprising beta-blocker in an amount that cause a less than 40% decrease in the amount of aMT6s in complete nocturnal urine.

2. A method of treating bruxism in an individual or a patient, said method comprising administering to said individual or said patient a composition comprising Bisoprolol, Nebivolol, Carvedilol, Acebutolol, Betaxolol, Bucindolol, Carteolol, Celiprolol, Esmolol, Labetalol, Metoprolol, Penbutolol, Pindolol, or Timolol.

3. A method of treating insomnia in an individual or a patient, said method comprising administering to said individual or said patient a composition comprising Bisoprolol, Nebivolol, Carvedilol, Acebutolol, Betaxolol, Bucindolol, Carteolol, Celiprolol, Esmolol, Labetalol, Metoprolol, Penbutolol, Pindolol, or Timolol.

4. The method of claim 3, wherein said insomnia is primary insomnia.

5. The method of claim 3, wherein said insomnia is secondary insomnia.

6. A method of treating a sleep disorder due to occasional stress in an individual or a patient, said method comprising administering to said individual or said patient a composition comprising Bisoprolol, Nebivolol, Carvedilol, Acebutolol, Betaxolol, Bucindolol, Carteolol, Celiprolol, Esmolol, Labetalol, Metoprolol, Penbutolol, Pindolol or Timolol.

7. The method of claim 3, wherein said insomnia is transient insomnia, intermittent insomnia, and/or chronic insomnia.

8. The method of claim 3, wherein said insomnia is treated in said individual or said patient suffering from stress, anxiety, or depression.

9. The method of claim 3, wherein said individual or said patient is an adult, an elderly individual, or a child.

10. The method of claim 3, wherein said composition is taken or inhaled at bedtime, after bedtime, when an insomnia episode is experienced, or less than 6 hours before bedtime.

11. The method of claim 3, wherein said composition comprises an amount of Bisoprolol, Nebivolol, Carvedilol, Acebutolol, Betaxolol, Bucindolol, Carteolol, Celiprolol, Esmolol, Labetalol, Metoprolol, Penbutolol, Pindolol, or Timolol, which is less than 51% of the lowest amount that is normally used for chronic treatment of hypertension, for treating said insomnia.

12. The method of claim 3, wherein said composition comprises an amount of Bisoprolol, Nebivolol, Carvedilol, Acebutolol, Betaxolol, Bucindolol, Carteolol, Celiprolol, Esmolol, Labetalol, Metoprolol, Penbutolol, Pindolol, or Timolol, which is less than 26% of the lowest amount that is normally used for chronic treatment of hypertension, for treating said of insomnia.

13. The method of claim 3, further comprising administering at least one compound or extract selected from the group consisting of Melatonin, any Melatonin receptor agonist, Valerian (Valeriana officinalis) extract, Doxylamine, Diazepam (Stesolid®), Oxazepam (Sobril®), Lorazepam (Temesta®), Alprazolam (Xanor®), Hydroxizin (Atarax®), Buspiron (Buspar®), Zopiclone (Imovane®), Zolpidem (Ambien®), Zaleplon (Sonata®), Ramelteon (Rozeram®), Eszopiclone (Lunesta®), Diphenhydramine (Benadryl®), Hydroxyzine (Atarax®), Flurazepam (Dalmane®), Quazepam (Doral®), Triazolam (Halcion®), Estazolam (ProSom®), Temazepam (Restoril®), Nitrazepam, Lormetazepam, and Acetaminophen (Tylenol®) for treating said insomnia.

14. The method of claim 1, wherein said individual or said patient is non-hypertensive.

15. The method of claim 2, wherein said individual or said patient is non-hypertensive.

16. The method of claim 3, wherein said individual or said patient is non-hypertensive.

17. The method of claim 6, wherein said individual or said patient is non-hypertensive.

18. The method of claim 3, wherein said Bisoprolol, Nebivolol, Carvedilol, Acebutolol, Betaxolol, Bucindolol, Carteolol, Celiprolol, Esmolol, Labetalol, Metoprolol, Penbutolol, Pindolol, or Timolol is administered in an amount that causes a less than 40% decrease in the amount of aMT6s in complete nocturnal urine.

19. The method of claim 1, wherein said beta-blocker is selected from the group consisting of Bisoprolol, Nebivolol, Carvedilol, Acebutolol, Betaxolol, Bucindolol, Carteolol, Celiprolol, Esmolol, Labetalol, Metoprolol, Penbutolol, Pindolol, and Timolol.
